(19) United States
(12) Patent Application Publication
(10) Pub. No.: US 2016/0206545 A1
(43) Pub. Date: Jul. 21, 2016

(54) EMULSIFIER-FREE, SKIN CONDITIONING, COSMETIC OR DERMATOLOGICAL PREPARATION WHICH CONTAINS PEELING ACTIVE INGREDIENTS

(71) Applicant: BEIERSDORF AG, Hamburg (DE)

(72) Inventor: Horst ARGEMBEAUX, Wentorf (DE)

(21) Appl. No.: 14/914,833

(22) PCT Filed: Aug. 25, 2014
(86) PCT No.: PCT/EP2014/067975

§ 371 Date: Feb. 26, 2016

(30) Foreign Application Priority Data
Aug. 29, 2013 (DE) 10 2013 217 242.7

(51) Int. Cl. A61K 8/85; A61K 8/02; A61K 8/20; A61K 8/34; A61K 8/97; A61K 8/25; A61Q 19/00; A61K 8/31

(52) U.S. Cl. CPC A61K 8/85; A61Q 19/00; A61K 8/0216; A61K 8/31; A61K 8/342; A61K 8/97; A61K 8/345; A61K 8/25; A61K 8/20; A61K 2800/30; A61K 2800/28; A61K 2800/33; A61K 2800/596; A61K 2800/412

(57) ABSTRACT

The invention relates to an emulsifier-free, skin conditioning cosmetic or dermatological preparation which contains peeling active ingredients.

The invention comprises an emulsifier-free, skin conditioning cosmetic or dermatological preparation with peeling active ingredients. The preparation is suitable for application to wet skin and thereby allows for rubbing in during showering and simultaneous peeling.

Rubbing in under wet conditions, skincare in the shower, is understood in summary as skin conditioning. This means inter alia:
1. use of a customary shower product for cleaning the skin, rinsing off
2. application/spreading of the preparation according to the invention on wet skin
3. renewed showering off with warm or cold water
4. drying off of the skin.

WO 2013064391 A2 describes cosmetic or dermatological preparations which allow rubbing in under the shower.

On account of a lack of time, many people desire the most efficient possible skincare and simultaneous skin protection.

Peeling or peel treatment is a cosmetic treatment in which the superficial layers of the skin are extensively removed.

During superficial peeling, the uppermost horny layer of the skin is removed by mechanical or chemical means. In colloquial language, this treatment is referred to as peeling, many suppliers refer to this method also as microdermabrasion.

The peeling agents added to the preparations are usually polyethylene, cooking salt, sea salt, sodium carbonate, sodium hydrogencarbonate, magnesium sulfate, magnesium chloride, sugar, clay earth, sand, plastic particles, powdered or ground kernels of walnut shells, apricot, peach or almond kernels.

Since the peeling agents are solid particulate substances which have an abrasive action, they have to be rinsed off together with the removed skin following application. For this purpose, the known peeling preparations are mostly provided with washing-active substances which ensure rinsing.

It is a disadvantage that the washing-active substances can dry the skin and skin damage may be the result.

It is desirable to provide a peeling preparation which can be used under the shower and which avoids the listed disadvantages.

It is desirable to provide a preparation which, on the one hand, is designed to be skin-compatible and, on the other hand, opens up the possibility of applying ingredients with abrasive action to the skin and can therefore be applied in a time-saving manner under the shower and/or to wet skin.

The invention is an emulsifier-free cosmetic or dermatological preparation comprising one or more polyacrylic acid polymers, one or more C14-22 fatty alcohols, and one or more waxes and/or a hydrocarbon mixture, and one or more peeling active ingredients.

Preferably, the preparation comprises waxes, in particular Cera Microcristallina, in the region of more than 0.5% by weight, in particular more than 13% by weight, based on the total mass of the preparation.

The preferred fraction refers both to the individual waxes and also preferably to the total amount of several waxes.

The peeling active ingredients (peeling bodies/peeling particles) are preferably selected from the group PG-10 stearate, polyethylene, cooking salt, sea salt, sodium carbonate, sodium hydrogencarbonate, magnesium sulfate, magnesium chloride, sugar, clay earth, sand, plastic particles, powdered or ground kernels of e.g. walnut shells, apricot, peach or almond kernels. Preferred peeling agents are to be selected from the group Himalaya salt, plastic particles made of viscose, cellulose, polypropylene, polyester, polyethylene terephthalate (PET), polytetrafluoroethylene (PTFE), aramid, nylon, Kevlar, polyvinyl derivatives, polyurethanes, polylactide, polyhydroxyalkanoate, polycarbonate, polystyrene, cellulose ester and/or polyethylene, as well as sparingly soluble or insoluble crystals such as e.g. calcium sulfate, calcium carbonate, encapsulated or nonencapsulated crystals such as e.g. calcium chloride, potassium chloride, magnesium chloride, sugar, silicates such as e.g. sea sand, hydrated silica, clay earth, slurries, shredded or ground natural products such as e.g. wheat, linseed, rice, corn, almonds, nuts, nut shells, pumpkin seeds, caraway, shredded or ground natural sponges such as e.g. loofah cucumbers, natural and synthetic waxes such as e.g. rice germ wax, carnauba wax, jojoba wax, beeswax.

Further preferably the peeling agents can be selected from the group hydrogenated castor oil, hydrated silica and/or polylactic acid (PLA), in each case preferably with a particle size of 50 to 500 μm.

Preferred peeling bodies with hydrogenated castor oil used are those which comprise castor oil, i.e. the oil obtained from Ricinus communis seeds which is then at least partially hydrogenated.

Preferably, these peeling bodies comprise at least 20% castor oil, preferably at least 50% castor oil and most preferably at least 90% castor oil, based on the total mass of the peeling body.

The degree of hydrogenation of the castor oil can be ascertained by determining the iodine number. Castor oils with an iodine number of 0 to 20 mg I₂/100 g have proven to be particularly suitable.

In principle, besides the hydrogenated castor oil, the peeling bodies can also comprise further substances, in particular waxes. Paraffin waxes, carnauba waxes, candelilla waxes, polyethylene waxes, oxidized polyethylene waxes or mixtures thereof are particularly suitable. Advantageously, those waxes are to be selected which have a melting range above 40° C., preferably above 80° C.

The peeling body is preferably produced by a process in which the hydrogenated castor oil, optionally with the addition of further substances, in particular further waxes, is melted and then the melt is dripped or sprayed on.

In principle, it is also possible to mix the peeling bodies based on hydrogenated castor oil with abrasive substances, for example with polyurethane abrasive bodies and then to add them to the preparations according to the invention.

According to the invention, the selection of the peeling active ingredients is advantageously to be selected from the group of lipophilic compounds.

Since the basic preparation according to the invention is composed of predominantly lipophilic substances, a longer stay on the skin can thus be realized.

The fraction of one or more peeling active ingredients is advantageously up to 25% by weight, based on the total mass of the preparation.

Surprisingly, peeling takes place with the preparation according to the invention and a care film remains on the skin after rinsing and drying on the skin.

The application of the combination of specific fatty alcohols, waxes and advantageously one or more film formers leads, after peeling, to skin protection and/or skincare even while showering.

The preparations according to the invention advantageously comprise one or more film formers.

These film formers are advantageously selected from the group hexadecene copolymer, trimethylsiloxysilicate, polypropylsilsesquioxane, polysilicone-25, acrylate copolymer, polyurethane, methacrylate, polyglyceryl stearate, dilinoleate crosspolymer, alkyl acrylate/methacrylic acid crosspolymer, IPDI copolymer, in particular VP/hexadecene copolymer, octyldodecyl citrate crosspolymer, trimethylsiloxysilicate/polypropylsilsesquioxane, polysilicone-25, ammonium acrylate copolymer, acrylate copolymer, polyurethane-2 and polymethyl methacrylate, polyglyceryl stearate/isostearate dilinoleate crosspolymer, octadecene/MA copolymer (and) methyl acetyl ricinoleate (and) diisooctyl adipate, trimethylsiloxysilicate, polyurethane-34, C8-22 alkyl acrylate/methacrylic acid crosspolymer and/or castor oil/IPDI copolymer.

The fraction of film formers is advantageously selected in the region of 0.1% by weight, in particular 0.5% by weight to 10% by weight, based on the total mass of the preparation.

In a comparison, the protective film that forms on the skin is investigated, once following the application of a rinse-off product with integrated skincare aspects (Nivea crème soft shower gel) on its own and once following application of the same product (Nivea crème soft shower gel) and subsequent application of the preparation according to the invention. After both applications, the skin is rinsed with water.

The measurements carried out with regard to refatting of the skin were made by IR imaging. The measurement technique is called IR-ATR (InfraRed-Attenuated Total Reflectance).

It was found that a protective film, visible by means of the mentioned measurement technique, remains on the skin and is detectable only following application of the preparation according to the invention. The demonstration takes place via the intensity of the hydrocarbon-IR bands (CH-IR bands).

The protective film according to the invention comprises a film which is formed on the skin.

The skin conditioning according to the invention comprises the rubbing in under wet conditions, in particular skincare while showering, where a protective film also remains on the skin after rinsing. The protective film can be detected by means of IR-ATR measurement technique and ideally has a thickness of at least 1 μm to 10 μm.

In particular, the skin conditioning according to the invention is characterized in that one or more active ingredients, one or more lipids and one or more skin-wetting agents are comprised and none of the substances that damage the skin barrier, in particular no emulsifiers and/or surfactants, are present in the protective film on the skin.

Only this film remaining on the skin permits the application and also the retention of active ingredients on the skin.

In the preparation according to the invention, one or more fatty alcohols and at least one additional wax and/or mixture of liquid and solid hydrocarbons, with a melting range from 5° C. to 75° C., preferably up to 55° C. (according to DSC), are combined, in particular melted.

I.e., advantageously, besides at least one fatty alcohol, at least one wax is present or besides fatty alcohol, at least one mixture of liquid and solid hydrocarbons is present. Ideally, fatty alcohol, wax and a mixture of hydrocarbons are present.

Advantageously, fatty alcohols to be selected are myristyl, cetearyl and/or stearyl alcohols, the waxes are Cera Microcristallina, coco glyceride, C18-36 acid triglyceride, synthetic wax, Cera Alba, paraffin, Copernicia Cerifera Cera, C18-38 alkyl hydroxystearoylstearate, Butyrospermum Parkii butter, olus oil, C20-40 alcohol and/or beeswax, and the hydrocarbon mixture is Paraffinum Liquidum.

Preference is given to Cera Microcristallina.

Preferred use concentrations of the wax or waxes is in the range from 0.5 to 20% by weight, based on the total mass of the preparation.

The fatty alcohols, waxes and hydrocarbon mixtures here all advantageously have a melting range from 5° C. to 75° C., preferably up to 55° C. (according to DSC).

DSC (differential scanning calorimetry) is a thermal method for measuring the amount of heat released/taken up by a sample during an isothermal procedure, heating or cooling (see DIN 53765, DIN 51007, ASTM E 474, ASTM D 3418). DSC is a comparative measurement method which permits the determination of amounts of heat of physical and chemical processes. If a material changes its physical state, such as e.g. melting or conversion of one crystalline form into another or if it reacts chemically, heat is taken up or released in the process. These amounts of heat can be measured quantitatively with the help of DSC. The method proceeds cyclically, such that, after the first heating curve, a defined cooling takes place and then the sample is heated once again in the given temperature range. Two kinds of information are thus obtained: in the first heating curve, all thermal effects including past history are evident. In the second heating curve, the past history has been eliminated and the pure thermal behavior of the sample can be evaluated under defined cooling conditions. The melting range of the fatty alcohols, waxes and hydrocarbons of between 4.5° C. and 75° C. according to DSC is the range determined in the first heating curve.

According to the invention, waxes that can be used are also fats and fat-like substances with a wax-like consistency. These include, inter alia, fats (triglycerides), mono- and diglycerides, natural and synthetic waxes, fat and wax alcohols, esters of fatty alcohols and fatty acids, and also fatty acid amides or any desired mixtures of this substance.

Particularly preferably, the waxes are selected from the group of fats, in particular from the group of natural waxes: Shorea Stenoptera seed butter, hydrogenated vegetable oil, hydrogenated coco glycerides, Butyrospermum Parkii butter, Theobroma Cacao (cocoa) seed butter, mango butter, hydrogenated palm kernel glycerides, hydrogenated palm glycerides, sunflower seed wax, soybean glycerides, Butyrospermum Parkii Unsaponifiables, wool wax, Cera Alba, beeswax, sugar cane wax, Cera Carnauba, candelilla wax, Japan wax, hydrogenated rapeseed oil, shellac wax, hydrogenated lecithin, hydrogenated soybean oil, from the group of synthetic waxes, in particular from:

Cera Microcristallina, synthetic beeswax, synthetic wax, polyethylene, paraffin wax, ceresin, ozokerite, from the group of fatty acids, in particular from:

palmitic acid, stearic acid, from the group of esters of fatty acids, in particular from:

cetearyl nonanoate, methyl palmitate, glyceryl tribehenate, glyceryl laurate, glyceryl stearate, cetyl palmitate; shea butter oleyl esters, PEG-8 beeswax.

In accordance with the invention, a distinction is to be made between the waxes which are preferably to be chosen as the peeling agent and the waxes of the preparation.

The fatty alcohols used are preferably C14 to C22 fatty alcohols. Preferably, the fatty alcohols are selected from the group of linear fatty alcohols, in particular myristyl alcohol (C₁₄H₃₀O), cetyl alcohol (or palmityl alcohol) (C₁₆H₃₄O), stearyl alcohol (or octadecyl alcohol) (C₁₈H₃₈O), and cetylstearyl alcohol (cetearyl alcohol), behenyl alcohol, lanolin alcohol, a mixture of the alcohols cetyl alcohol (hexadecanol) and stearyl alcohol (octadecanol).

The fraction of C14-22 fatty alcohols overall is advantageously 0.5 to 14% by weight, in particular 7 to 9% by weight, or in particular 0.5-5%, based on the total mass of the preparation.

The hydrocarbon mixtures used are preferably hydrocarbon gels or mixtures of liquid and solid paraffin hydrocarbons. Preferably, the content of solid hydrocarbons in the hydrocarbon mixture is between 1 and 50%, particularly preferably between 10 and 30%. The use of hydrocarbon mixtures which form fringed micelles or paracrystalline structures is advantageous.

The fraction of the hydrocarbon mixture overall is advantageously 1 to 50% by weight, in particular 20 to 30% by weight, based on the total mass of the preparation.

According to the invention, fatty alcohol, in particular two or three fatty alcohols, are obligatorily present in the preparation. Additionally, one or more waxes are added to the preparation. Preferably, instead of the wax, it is also possible to add a hydrocarbon mixture of hydrocarbons that are liquid and solid at room temperature.

Ideally, the preparation comprises all three building blocks, fatty alcohols, waxes and hydrocarbon mixture.

Cosmetic or dermatological substances known to the person skilled in the art can then be added to the production and preparation according to the invention, in which case their addition must not adversely affect the skin-conditioning properties and the peeling of the obtainable preparation.

Thickeners, fillers and neutralizing agents are advantageously added to the preparations.

Thickeners are advantageously suitable for stabilizing the system and boost the skin-conditioning properties and the special skin feel of the preparations according to the invention.

The filler added is preferably aluminum starch octenylsuccinate, which likewise leads to an optimization of the skin feel in that the skin protective film acts somewhat more velvety.

The neutralizing agent added is advantageously aqueous sodium hydroxide, so that the thickeners can form their gel network and a stable system is formed.

The preparation according to the invention is emulsifier-free, i.e., in accordance with the invention, the polyacrylic acid polymers or peeling agents which may have an emulsifying effect are not taken to be emulsifiers.

In other words, besides the polyacrylic acid polymers and/or peeling agents, no further emulsifiers are added to the preparation.

Emulsifier-free also encompasses a minimum content of additional emulsifiers of less than 1% by weight, based on the total mass of the preparation, which may be present, for example, as a result of contaminations or entrainments. The influence on the product performance in these quantitative ranges is insignificant if appropriate.

However, preference is given to a content of emulsifiers of 0% by weight.

The polyacrylic acid polymers are understood as being the polymers of acrylic acid and/or methacrylic acid, and acrylate crosspolymers known in cosmetics.

Preferably, these are polymers (macromolecules) with a high molecular weight (>1 mg/mol) which consist of a backbone of polyacrylic acid and small amounts of polyalkenyl ether crosslinkages. They are also referred to as carbomers. These water-soluble or dispersible polymers can bring about a significant increase in viscosity in the liquid in which they are dissolved or dispersed. This is brought about by the formation of carbomer microgels in the water.

Besides the carbomers, particularly preferred polyacrylic acid polymers are those acrylate crosspolymers which exert a polymeric emulsifier effect.

Polymeric emulsifiers are primarily polyacrylic acid polymers with a high molecular weight. These emulsifying polyacrylic acid polymers have a small lipophilic fraction in addition to the hydrophilic main part. In the context of the present invention, very particular preference is given to acrylate crosspolymers which have the INCI name “Acrylates/C10-30 Alkyl Acrylate Crosspolymer” and are obtainable under the trade names Pemulen TR-1 and Pemulen TR-2, and also Carbopol 1342, Carbopol 1382 and Carbopol ETD 2020 from NOVEON.

The polyacrylic acid polymers are particularly preferably selected from the group of acrylates/C10-30 alkyl acrylate crosspolymers and/or carbomers. Particular preference is given to acrylates/C10-30 alkyl acrylate crosspolymer Pemulen® TR-1, e.g. from Lubrizol and Carbopol® 3128 from Lubrizol.

A specific combination of polyacrylic acid polymers with an emulsifying effect, such as the Pemulen TR-1 with other polyacrylic acid polymers, such as Carbopol 3128, which improve the sensory properties and ensure the stability of the preparation, especially at higher temperatures, and a compound with free water is in accordance with the invention here.

Particular preference here is given to a combination of three polyacrylic acid polymers, where one polyacrylic acid polymer has an emulsifying effect, such as e.g. the Pemulen TR-1 or Pemulen TR-2, with other polyacrylic acid polymers which improve the sensory properties and ensure the stability of the preparation, especially at higher temperatures (e.g. Carbopol 3128) and a polyacrylic acid polymer which improve the sensory properties upon absorption of free water (e.g. Carbopol 981).

The preparation according to the invention advantageously therefore comprises preferably at least three polyacrylic acid polymers, in particular three polyacrylic acid polymers which differ in their properties.

The fraction of polyacrylic acid polymers overall is preferably 0.05 to 2% by weight, in particular 0.2 to 1% by weight, based on the total mass of the preparation.

The combination of at least two polyacrylic acid polymers with at least two C14-22 fatty alcohols has proven to be advantageous for the improved stabilization of the preparation and in particular the skin feel when used on damp/wet skin is not unpleasant, nonwaxy, harsh or squeaky.

According to the invention, two polyacrylic acid polymers or three polyacrylic acid polymers are to be understood such that in each case one polyacrylic acid polymer differs from the others in each case in at least one property. The substance group acrylates/C10-30 alkyl acrylate crosspolymers includes, for example, the commercial products Pemulen TR-1 and TR-2.

Carbomers differ for example in types A, B and C. Differences are herein for example their gels with different viscosities (United States Pharmacopoeia, USP).

Moreover, a fraction of waxes or preferably a mixture of liquid and solid hydrocarbons with a melting range from 4.5 to 75° C., in particular up to 55° C. according to DSC is essential to the invention and therefore preferred.

Nonpolar to medium-polar lipids can optionally advantageously be added as oils to the preparations according to the invention. Otherwise, the stability is more difficult to establish on account of the freedom from emulsifiers.

In the context of the present disclosure, the generic term used for fats, oils, waxes and the like is the expression “lipids”, as is entirely familiar to the person skilled in the art. The terms “oil phase” and “lipid phase” are also used synonymously.

Oils and fats differ inter alia in their polarity. It is proposed to adopt the interfacial tension towards water as a measure of the polarity index of an oil or of an oil phase. This means that the polarity of the oil phase in question is greater, the lower the interfacial tension between this oil phase and water is. According to the invention, the interfacial tension is considered to be a possible measure of the polarity of a given oil component.

The interfacial tension is any force which acts on an imaginary line, one meter in length, in the interface between two phases. The physical unit for this interfacial tension is classically calculated according to the force/length relationship and is usually given in mN/m (millinewtons divided by meters). It has a positive sign if it endeavors to make the interface smaller. In the reversed case, it has a negative sign.

The preparation according to the invention permits for the first time the application of peeling and simultaneous care while showering, and also, if appropriate, the application of active ingredients even during the showering process.

The preparations according to the invention are advantageously only formulated with preservatives which have a solubility in water of more than 0.75% at 20° C. On account of the lack of emulsifiers, destabilizations and crystallization may otherwise result.

The preparations according to the invention are furthermore preferably also free from surfactants.

Surfactants are substances which reduce the surface tension of a liquid or the interfacial tension between two phases and permit or assist the formation of dispersions. Surfactants enable two liquids that are actually immiscible with one another, such as, for example, oil and water, to be dispersed.

Furthermore, surfactants are described as amphiphilic substances which are able to dissolve organic, nonpolar substances in water. As a consequence of their specific molecular structure with at least one hydrophilic and one hydrophobic molecular moiety, they provide for a reduction in the surface tension of the water, the wetting of the skin, the facilitation of soil removal and dissolution, easy rinsing off and—according to desires—foam regulation.

The hydrophilic moieties of a surfactant molecule are mostly polar functional groups, for example —COO⁻, —OSO₃²⁻, —SO₃⁻, whereas the hydrophobic moieties are generally nonpolar hydrocarbon radicals. Surfactants are generally classified according to type and charge of the hydrophilic molecular moiety. In this connection, four groups can be differentiated:
anionic surfactants,
cationic surfactants,
amphoteric surfactants and
nonionic surfactants.

Anionic surfactants generally have carboxylate, sulfate or sulfonate groups as functional groups. In aqueous solution, they form negatively charged organic ions in an acidic or neutral medium. Cationic surfactants are characterized virtually exclusively by the presence of a quaternary ammonium group. In aqueous solution, they form positively charged organic ions in the acidic or neutral medium. Amphoteric surfactants contain both anionic and cationic groups and behave accordingly in aqueous solution as anionic or cationic surfactants depending on the pH. In a strongly acidic medium, they have a positive charge and in an alkaline medium, a negative charge.

Furthermore, detersive substances are known, such as, for example, cationic surfactants, in particular quaternary ammonium compounds. A detersive substance is used in detergents, washing up compositions, shampoos, shower gels and refers to the fraction of the formulation which influences the washing or cleaning performance. Detersive substances increase the “solubility” of fat and grease particles in water which adhere in the laundry or on the body. They can be of natural or synthetic origin. They are divided according to their type of charge into anionic, cationic, amopholytic or nonionic.

Emulsifiers enable two liquids that are immiscible with one another (for example oil in water) to combine to give an emulsion. On account of the amphiphilic character, they penetrate into the oil with their fat-soluble moiety. As a result of the hydrophilic moiety, the oil droplet produced by stirring can then be dispersed in the aqueous environment. Emulsifiers primarily have no washing active surfactant character.

Emulsifiers and surfactants can damage the barrier layer of the skin. For this reason, neither emulsifiers nor surfactants are added to the preparations, i.e. the addition of additional washing-active substances is advantageously dispensed with.

Advantageously, skin-wetting agents, moisturizers, are added to the preparations.

The preparations are used on wet skin and in particular also for shaving.

The preparation according to the invention can be used for conditioning the skin.

It permits the generation of a skin protective film following application of the preparation to the skin and subsequent rinsing with water.

The protective film which is formed is ideally at least 1 μm thick (measured according to IR-ATR measurement technique) and/or comprises no substances that are harmful to the skin barrier, in particular no emulsifiers, surfactants, PEGs and/or organohalogen compounds.

Furthermore, cosmetic active ingredients may be present in the preparations according to the invention.

Preferred embodiments of the preparations according to the invention comprise one or more waxes, a hydrocarbon mixture and an active ingredient from the group of the skin-wetting agents, where the fatty alcohols selected are myristyl, cetearyl and/or stearyl alcohols, the wax selected is Cera Microcristallina and the hydrocarbon mixture selected is Paraffinum Liquidum.

The skin-wetting active ingredient to be selected advantageously in this preparation is glycerol, as well as ubiquinone active ingredient Q10 for skincare and a cooling active ingredient such as menthol.

This three-way combination of active ingredients in combination with a peeling agent brings about a pleasant skin feel and care both during showering and also a long-lasting skincare.

In the event of limitations to substances specified as preferred, be they lipids, waxes, peeling agents, active ingredients or film formers or further constituents specified as being preferred, then their preferred fraction ranges thus also refer to the individual constituents then selected. The other constituents excluded by the limitation then no longer count towards the listed fraction ranges.

The examples below illustrate the preparation according to the invention in order to obtain preparations according to the invention.

The numerical values are fractions by weight, based on the total mass of the preparation.

### EXAMPLES

| Ingredient | 1 | 2 | 3 | 4 | 5 |
|---|---|---|---|---|---|
| Cera Microcristallina | 25.000 | 16.5000 | 16.5000 | 35.000 | 45.000 |
| Paraffinium Liquidum | | 8.5000 | 8.5000 | | |
| Myristyl alcohol | 1.0000 | 1.0000 | 1.0000 | 2.0000 | |
| Cetearyl alcohol | 5.0000 | 5.0000 | 5.0000 | 4.0000 | 4.0000 |
| Stearyl alcohol | 2.0000 | 2.0000 | 2.0000 | 3.0000 | 3.0000 |
| Hydrogenated coco | 3.0000 | 3.0000 | 3.0000 | 2.0000 | 2.0000 |
| glycerides | | | | | |
| Almond oil | | | 0.3500 | | 0.7000 |
| Aluminum starch | 1.0000 | 1.0000 | 1.0000 | 1.0000 | 1.0000 |
| octenyl succinate | | | | | |
| Perfume | 0.8000 | 0.7000 | 1.0000 | 0.7000 | 0.7000 |
| Glycerol | 5.1000 | 5.1000 | 5.1000 | 15.100 | 10.100 |
| Sodium hydroxide | 0.1600 | 0.1600 | 0.1600 | 0.1600 | 0.1600 |
| solution 45% strength | | | | | |
| Phenoxyethanol | 0.5000 | 0.5000 | 0.5000 | 0.4000 | 0.4000 |
| Methylisothiazolinones | 0.0900 | 0.0900 | 0.0900 | 0.0800 | 0.0800 |
| Acrylates/C10-30 alkyl | 0.1000 | 0.1000 | 0.1000 | 0.1200 | |
| acrylate crosspolymer | | | | | |
| (Carbopol 3128) | | | | | |
| Carbomer | 0.0200 | 0.0200 | 0.0200 | 0.0200 | 0.0200 |
| (Carbopol 981) | | | | | |
| Acrylates/C10-30 alkyl | 0.1000 | 0.1000 | 0.1000 | 0.1200 | 0.1400 |
| acrylate crosspolymer | | | | | |
| (Pemulen TR-1) | | | | | |
| Q10 | | | 0.0045 | | |
| Menthol | | | 0.3000 | | |
| Sea salt | 0.0100 | 0.0100 | | 0.0100 | 0.0500 |
| Hydrogenated castor oil | 10.0000 | | | | 25.000 |
| (50% strength) in | | | | | |
| paraffin wax | | | | | |
| PLA | 0.5000 | | | 1.600 | |
| Silica | | 2.450 | 0.800 | | |
| Water | ad 100 | ad 100 | ad 100 | ad 100 | ad 100 |

1.-13. (canceled)

14. A cosmetic or dermatological preparation, wherein the preparation is emulsifier-free and comprises (i) one or more polyacrylic acid polymers, (ii) one or more C14-22 fatty alcohols, (iii) one or more waxes and/or a hydrocarbon mixture, and (iv) one or more peeling active ingredients.

15. The preparation of claim 14, wherein the preparation comprises more than 0.5% by weight of one or more waxes, based on a total weight of the preparation.

16. The preparation of claim 14, wherein (ii) comprises one or more of myristyl alcohol, cetearyl alcohol, stearyl alcohol.

17. The preparation of claim 14, wherein the one or more waxes comprise one or more of Cera Microcristallina, coca glyceride, C18-36 acid triglyceride, synthetic wax, Cera Alba, paraffin, Copernicia Cerifera Cera, C18-38 alkyl hydroxystearoylstearate, Butyrospermum Parkii butter, olus oil, C20-40 alcohol, beeswax.

18. The preparation of claim 14, wherein the preparation further comprises one or more film formers.

19. The preparation of claim 18, wherein the one or more film formers are selected from one or more of hexadecene copolymer, trimethylsiloxysilicate, polypropylsilsesquioxane, polysilicone-25, acrylate copolymer, polyurethane, methacrylate, polyglyceryl stearate, dilinoleate crosspolymer, alkyl acrylate/methacrylic acid crosspolymer, IPDI copolymer.

20. The preparation of claim 18, wherein the one or more film formers are selected from one or more of VP/hexadecene copolymer, octyldodecyl citrate crosspolymer, trimethylsiloxysilicate/polypropylsilsesquioxane, polysilicone-25, ammonium acrylate copolymer, acrylate copolymer, polyurethane-2, polymethyl methacrylate, polyglyceryl stearate/isostearate dilinoleate crosspolymer, octadecene/MA copolymer (and) methyl acetylricinoleate (and) diisooctyl adipate, trimethylsiloxysilicate, polyurethane-34, C8-22 alkyl acrylate/methacrylic acid crosspolymer, castor oil/IPDI copolymer.

21. The preparation of claim 18, wherein the one or more film formers are selected from one or more of castor oil/IPDI copolymer, trimethylsiloxysilicate/polypropylsilsesquioxane, acrylate copolymer.

22. The preparation of claim 18, wherein the one or more film formers are present in a concentration of from 0.1% to 10% by weight, based on a total weight of the preparation.

23. The preparation of claim 22, wherein the one or more film formers are present in a concentration of at least 0.5% by weight, based on a total weight of the preparation.

24. The preparation of claim 14, wherein (iv) comprises hydrogenated castor oil.

25. The preparation of claim 14, wherein (iv) comprises hydrated silica.

26. The preparation of claim 14, wherein (iv) comprises polylactic acid (PLA).

27. The preparation of claim 14, wherein the one or more peeling active ingredients have a particle size in a range from 50 μm to 500 μm.

28. The preparation of claim 14, wherein the preparation further comprises one or more skin-wetting agents.

29. The preparation of claim 14, wherein the preparation is free of surfactants.

30. The preparation of claim 14, wherein the preparation comprises up to 25% by weight of (iv), based on a total weight of the preparation.

31. The preparation of claim 14, wherein (iii) comprises one or more waxes and a hydrocarbon mixture.

32. The preparation of claim 31, wherein (iii) comprises cera microcristallina.

33. A method of applying one or more peeling active ingredients onto skin, wherein the method comprises applying the preparation of claim 14 onto wet skin.
